(19) United States
(12) Patent Application Publication
(10) Pub. No.: US 2020/0205656 A1
(43) Pub. Date: Jul. 2, 2020

(54) MULTI-FACTOR CONTROL SYSTEM FOR OPHTHALMIC REFRACTION MODIFICATION

(71) Applicant: RxSight, Inc., Aliso Viejo, CA (US)

(72) Inventors: Matt Haller, Costa Mesa, CA (US); Ronald M. Kurtz, Irvine, CA (US); Gergely T. Zimanyi, Berkeley, CA (US)

(73) Assignee: RxSight, Inc., Aliso Viejo, CA (US)

(21) Appl. No.: 16/237,659

(22) Filed: Dec. 31, 2018

(51) Int. Cl. A61B 3/103; A61B 3/10; G16H 50/20; A61B 5/00; A61B 3/028; A61F 9/008

(52) U.S. Cl. CPC A61B 3/103; A61B 3/102; A61B 3/1015; G16H 20/40; A61B 5/4836; A61B 3/028; A61F 9/00804; G16H 50/20

(57) ABSTRACT

An ophthalmic system comprises a Refraction Modification System for modifying a refraction of an eye, in response to refraction modification parameters; and a Prescription Engine, for generating the refraction modification parameters for the Refraction Modification System by a Prescription Algorithm that combines one or more diagnostic factor, determined by an ophthalmic diagnostic device, and one or more patient factor. Correspondingly, a method of operating an ophthalmic system comprises receiving one or more diagnostic factor by a Prescription Engine, determined by an ophthalmic diagnostic device; receiving one or more patient factor by the Prescription Engine; and generating refraction modification parameters by the Prescription Engine for a Refraction Modification System by combining the one or more diagnostic factor and the one or more patient factor with a Prescription Algorithm; wherein the Refraction Modification System is configured for modifying a refraction of an eye, in response to the generated refraction modification parameters.

### TECHNICAL FIELD

This invention relates to ophthalmic refraction modification systems, and more specifically to multi-factor control systems for ophthalmic refraction modification.

### BACKGROUND

The techniques of cataract surgery are progressing at an impressive pace. Generations of phacoemulsification platforms and more recently introduced surgical lasers keep increasing the precision of the placement of intraocular lenses (IOLs) and keep reducing unintended medical outcomes. Nevertheless, after the IOLs have been implanted, the postsurgical healing process can shift or tilt the IOLs in a notable fraction of the patients, leading to a diminished visual acuity, and a deviation from the planned surgical outcome.

A new technique has been developed recently to correct or mitigate such a postsurgical IOL shift or tilt. The new technique is capable of adjusting the optical properties of the IOLs with a postsurgical procedure to compensate the shift or tilt of the IOL. As described in commonly owned U.S. Pat. No. 6,905,641, to Platt et al, entitled: “Delivery system for post-operative power adjustment of adjustable lens”, hereby incorporated by reference in its entirety, the IOLs can be fabricated from a photo-polymerizable material, henceforth making them Light Adjustable Lenses, or LALs. In the days after the surgery, the implanted LALs may shift and tilt, eventually settling into a postsurgical position different from what the surgeon planned. Once the LAL settled, a Light Delivery System (LDD) can be used to illuminate the LALs with an illumination pattern that induces photopolymerization, thus changing the refractive properties of the LALs. This refractive change adjusts the LAL optical performance to compensate the unintended postsurgical shift or tilt of the LAL.

In order to maximize the medical benefits of this Light Adjustment procedure of the LALs, the surgeon needs to specify the details and parameters of the illumination, including its beam diameter, power, duration and spatial profile, among others. In some approaches, the surgeon may rely on objectively determined diagnostic factors, e.g., a measured optical power and a cylinder. In other approaches, the surgeon may ask the patient for subjective vision preferences, such as how much relative value the patient attributes to optimizing the visual acuity for the distance vision versus for the near vision. However, how to combine the objective and the subjective factors is a largely undeveloped field. Therefore, there remains a medical need for practices and techniques to incorporate both objective, diagnostic factors and subjective patient factors into the eventual selection of the illumination parameters. While motivated from the specific technology of Light Adjustable Lenses, this medical need is quite broad, as the same unmet need exists in a variety of refraction modification systems.

### SUMMARY

The above described medical needs are addressed by embodiments of ophthalmic systems, comprising: a Refraction Modification System for modifying a refraction of an eye, in response to refraction modification parameters; and a Prescription Engine, for generating the refraction modification parameters for the Refraction Modification System by a Prescription Algorithm that combines one or more diagnostic factor, determined by an ophthalmic diagnostic device, and one or more patient factor.

In other embodiments, a method of operating an ophthalmic system comprises receiving one or more diagnostic factor by a Prescription Engine, determined by an ophthalmic diagnostic device; receiving one or more patient factor by the Prescription Engine; and generating refraction modification parameters by the Prescription Engine for a Refraction Modification System by combining the one or more diagnostic factor and the one or more patient factor with a Prescription Algorithm; wherein the Refraction Modification System is configured for modifying a refraction of an eye, in response to the generated refraction modification parameters.

### BRIEF DESCRIPTION OF THE DRAWINGS

FIGS. 1A-B illustrate a modular and an integrated ophthalmic system 100.

FIG. 2 illustrates a Prescription Engine 120.

FIG. 3 illustrates diagnostic factors 140 and diagnostic devices 145.

FIGS. 4A-B illustrate patient factors 150, patient visual tests 155 and physician control factors 160.

FIG. 5 illustrates patient factors 150 and patient visual tests 155.

FIG. 6 illustrates physician control factors 160.

FIG. 7 illustrates refraction modification parameters 130.

FIG. 8 illustrates Refraction Modification Systems 110.

FIGS. 9A-C illustrate refraction modification parameters 130 in relation to a LAL 200.

FIG. 10 illustrates a method 300 of controlling an ophthalmic system 100.

### DETAILED DESCRIPTION

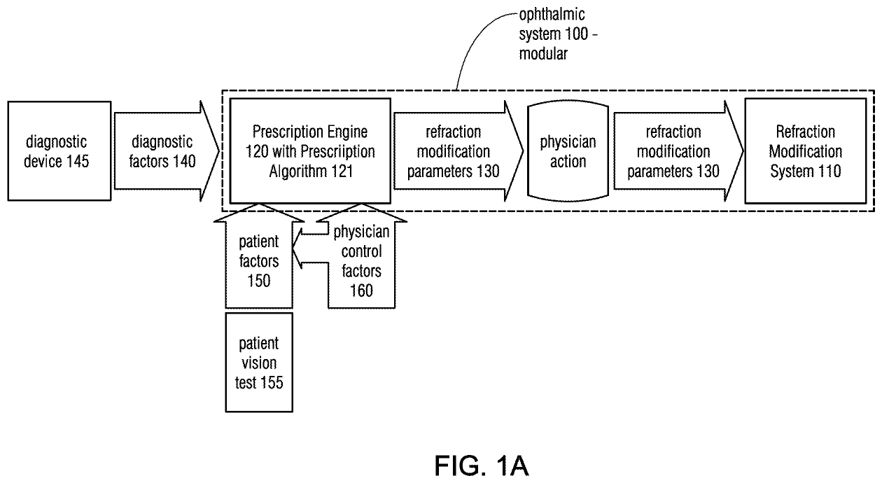

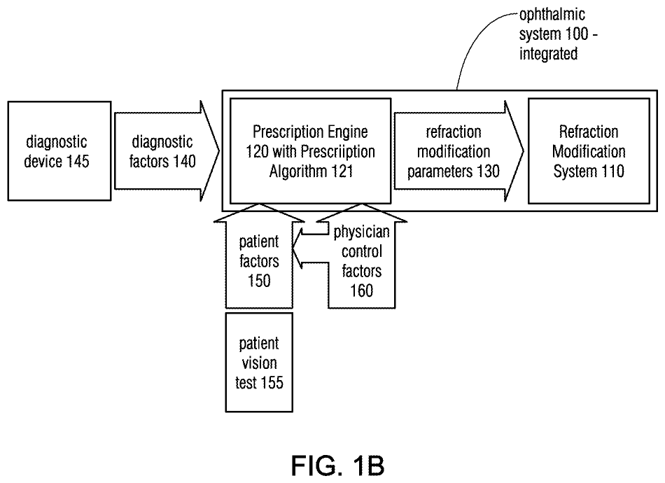

FIG. 1A illustrates an ophthalmic system 100 that includes a Refraction Modification System 110 for modifying a refraction of an eye, in response to refraction modification parameters 130; and a Prescription Engine 120, for generating the refraction modification parameters 130 for the Refraction Modification System 110 by a Prescription Algorithm 121 that combines one or more diagnostic factor 140, determined by an ophthalmic diagnostic device 145, and one or more patient factor 150.

FIG. 1A shows a modular embodiment of the ophthalmic system 100, in the sense that the Prescription Engine 120 is separate from the Refraction Modification System 110 and does not directly communicate with it. This is referenced by grouping these modules 110 and 120 together only by the dashed line. In these modular ophthalmic systems 100, the Prescription Engine 120 can be configured for outputting the refraction modification parameters 130; while the Refraction Modification System 110 can be configured for receiving the refraction modification parameters 130 at least partially through a physician action. In some cases, the Prescription Engine 120 can display the refraction modification parameters 130 on a display, monitor, or graphic interface. In response, the physician can make a decision based on the displayed and thus suggested refraction modification parameters 130, further based on her own diagnosis of the patient and her judgement, and then select and enter the refraction modification parameters 130 into a terminal, keyboard, touchscreen, or control panel of the Refraction Modification System 110. The physician is free to enter the displayed/suggested refraction modification parameters 130, or to modify them as she sees fit. In other embodiments, the Prescription Engine 120 can output the refraction modification parameters 130 as a paper printout, a printed barcode, or an electronically displayed printout or barcode, and then the physician can scan the barcode into the Refraction Modification System 110, or she can insert the printout into a machine vision system of the Refraction Modification System 110. In yet other embodiments, the Prescription Engine 120 can output the refraction modification parameters 130 via an electronic data device, such as a flash drive, a thumb drive, an RF-transmissive device, a blue-tooth device, a wireless device, or a memory device. Any of these memory devices can then be inserted into an input terminal of the Refraction Modification System 110 by the physician, optionally followed by a modification step.

FIG. 1B illustrates another, integrated embodiment of the ophthalmic system 100 in the sense that the Prescription Engine 120 is configured for coupling the refraction modification parameters 130 into the Refraction Modification System 110 more directly. In exemplary embodiments, the coupling can be an electronic, opto-electronic or blue-tooth connection, such that the refraction modification parameters 130 are communicated by the Prescription Engine 120 to the Refraction Modification System 110 without necessitating a physician action. In some of these embodiments, it may still be a requirement that the Refraction Modification System 110 accepts the refraction modification parameters 130 as proper input only after a physician validated them, or in some other form controlled the communication. Also, in some embodiments, the physician may be invited to possibly modify the refraction modification parameters 130 after they have been coupled by the Prescription Engine 120 into the Refraction Modification System 110. Some embodiments of these integrated ophthalmic systems may in fact house the Prescription Engine 120 and the Refraction Modification System 110 in a single, integrated housing.

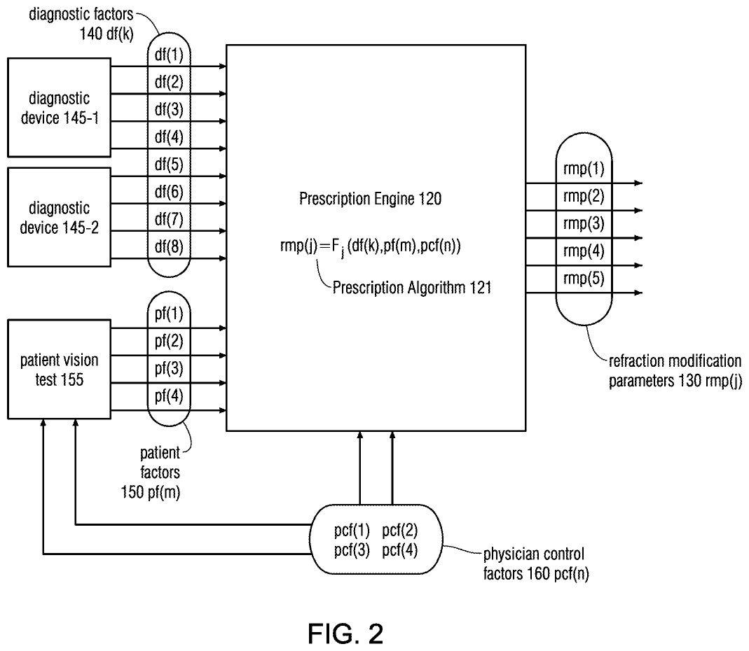

FIG. 2 illustrates embodiments of the Prescription Engine 120 in some detail. The diagnostic factors 140 can be represented as elements of a diagnostic factor vector 140 df(k), labeled by the index k. The elements of the diagnostic factor vector 140 df(k) can originate from one or more diagnostic devices 145, e.g. the shown two diagnostic devices 145-1 and 145-2. The patient factors 150 can be represented as elements of a patient factor vector 150 pf(m), that were generated by a patient vision test 155. In some embodiments, additional physician control factors 160, forming a physician control factor vector 160 pcf(n), can be also entered into the Prescription Engine 120, or can impact the results of the patient vision test 155, as described later. The number of components of these vectors df(k), pf(m), and pcf(n) can be different.

The Prescription Engine 120 can be embodied as a programmable computer, a dedicated processor, an application specific integrated circuit (ASIC), or as a self-standing electronic device. In these embodiments, the Prescription Engine 120 has one or more input terminals, in order to receive the inputted vectors df(k), pf(m), and pcf(n). These input terminals can be electronic, optical, or a graphical user interface, among others. In some instructive embodiments, the Prescription Engine 120 can be implemented as an iPad, or any other portable or mobile electronic device. The Prescription Engine 120 can be implemented as a locally deployed system, or it can be deployed in the cloud, on board of a remote server. In any of these embodiments, the Prescription Engine 120 can involve a Prescription Algorithm 121 that takes the elements of the inputted df(k), pf(m), and optionally the pcf(n) vectors, and determines from these inputs the refraction modification parameters 130 that can be elements of a vector rmp(j). In a general sense, the Prescription Algorithm 121 accepts two or three vectors as input, and generates a responsive vector as an output:

rmp(j)=Fⱼ(df(k),pf(m),pcf(n)). (1)

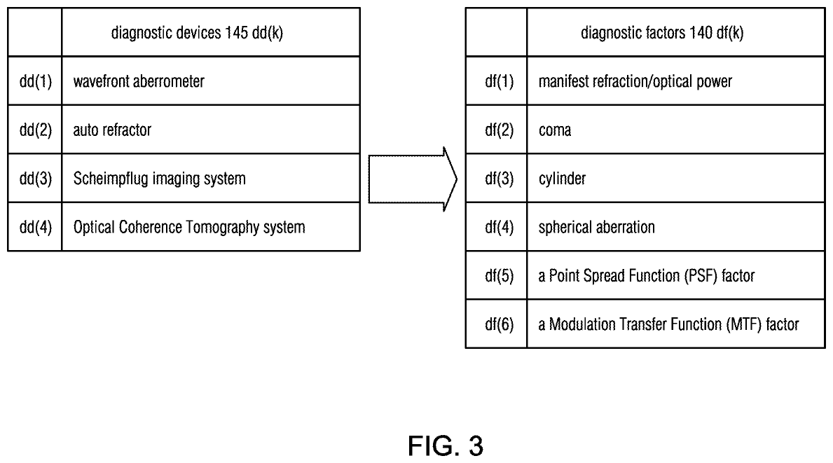

FIG. 3 illustrates that the diagnostic device 145 can be a wide variety of ophthalmic devices, for example, a wavefront aberrometer, an auto refractor, a Scheimpflug imaging system, or an Optical Coherence Tomography system.

These diagnostic devices 145 can determine a variety of objective diagnostic factors 140 df(k), for example, a manifest refraction or optical power df(1), a coma df(2), a cylinder df(3), a spherical aberration df(4), a Point Spread Function factor df(5), or a Modulation Transfer Function factor df(6) in an objective manner, not asking for the patient's subjective input.

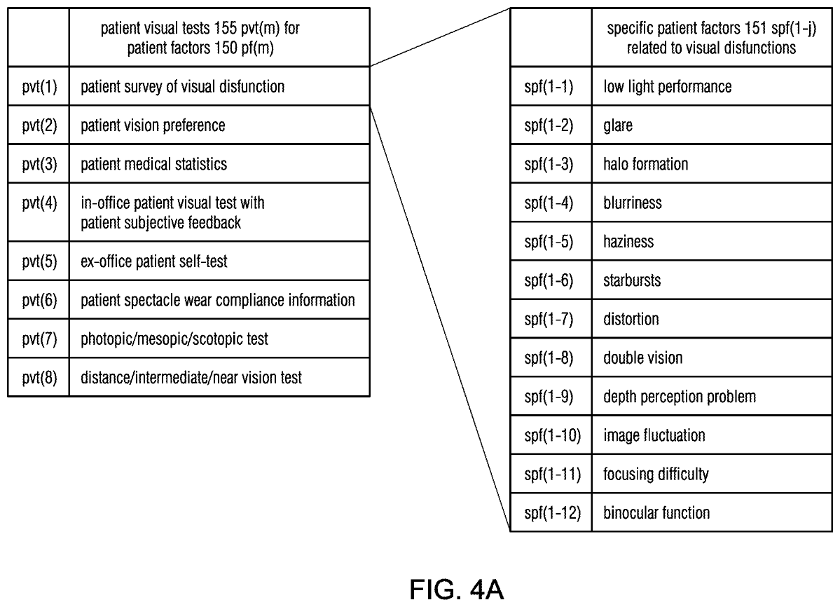

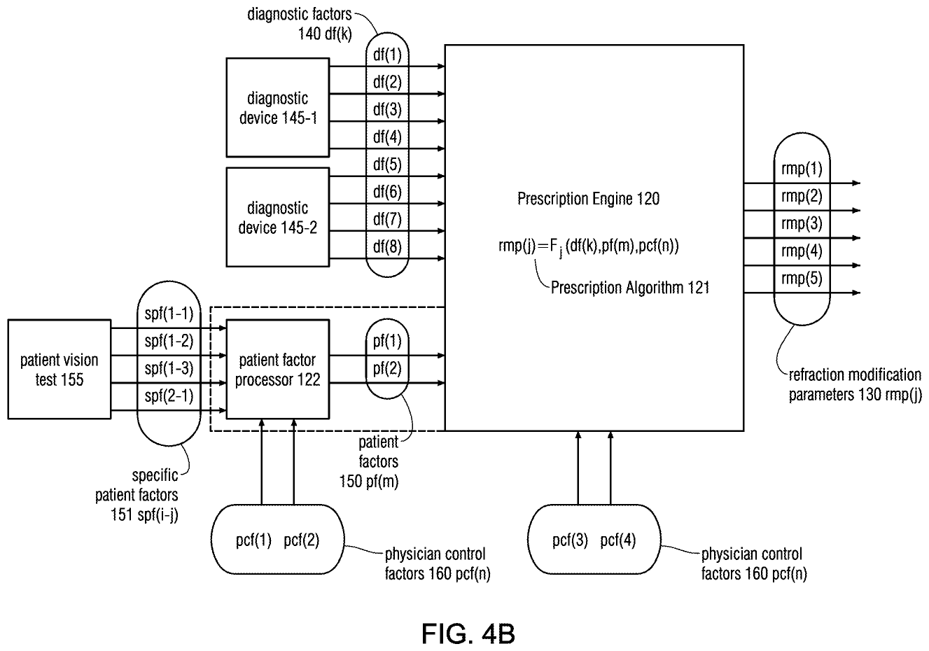

FIG. 4A, left panel illustrates that the dominantly subjective patient factors 150 pf(m) can be determined by one or more patient vision test 155 pvt(m), including a patient survey of visual disfunctions pvt(1); a patient vision preference pvt(2); a patient medical statistics pvt(3); an in-office patient visual test involving patient subjective feedback, such as a vision test using a phoropter, pvt(4); an ex-office patient self-test, e.g. using an application on a mobile personal electronic device at home, possibly over an extended period, pvt(5); a patient spectacle wear compliance information pvt(6); a photopic test, a mesopic test, and a scotopic test pvt(7); and a distance vision test, intermediate vision test, or near vision test, possibly using a phoropter and patient subjective feedback pvt(8). Here the term “patient vision test” is used in a broad sense, as a protocol for acquiring information about the patient's vision. Besides using patient feedback-utilizing diagnostic tools, embodiments of the patient vision tests pvt(m) can include verbal discussions with the patient, asking to fill out patient surveys, and in general asking the patient to provide information related to his/her vision. Visible from these embodiments of the patient vision tests 155 pvt(m), the patient factors 150 pf(m) capture substantially subjective information about or from the patient, while they can also make use of objective measurement devices like a phoropter. The relative weight of the subjective and objective component of the patient information is thus different for different patient factors 150 pf(m).

In some embodiments, the determination of the patient factors 150 pf(m) can be impacted by one or more physician control factors 160, adjustable by a physician, as discussed next.

One of the common aspects of these patient visual tests 155 pvt(m) of determining the patient factors 150 pf(m) is that they capture at least partially subjective information about the vision of the patient. These subjectively-determined patient factors 150 pf(m) can provide information complementary to the objective diagnostic factors 140 df(k), determined by the objective diagnostic devices 145. Given the complexity of the overall human visual acuity, a distinct advantage of the ophthalmic system 100 is that it combines the dominantly objective diagnostic factors 140 df(k) with the dominantly subjective patient factors 150 pf(m) to qualitatively better assist the physician's planning of the eventual refraction modification.

FIG. 4A, right panel shows that embodiments of the patient survey of visual disfunctions pvt(1) can determine patient factors 150 pf(m) that can be either specific patient factors 151 spf(i-j), or a combination of several specific patient factors 151 spf(i-j). Examples of specific patient factors 151 spf(i-j) include a patient's response to a patient survey of visual disfunctions pvt(1). As a shorthand, these specific patient factors 151 spf(1-j) will be referenced with the visual disfunction the patient characterized in the survey response. With this convention, examples of the specific patient factors 151 spf(i-j) include the patient's characterization of the following visual disfunctions the patient is experiencing: a low light performance spf(1-1), a glare spf(1-2), a halo formation spf(1-3), a blurriness spf(1-4), a haziness spf(1-5), starbursts spf(1-6), various distortions spf(1-7), a double vision spf(1-8), a depth perception and related problems spf(1-9), an image fluctuation spf(1-10), a focusing difficulty spf(1-11), and a binocular function spf(1-12).

Examples of these visual disfunctions, and corresponding specific patient factors spf(1-1)-spf(1-12) are described and discussed in notable detail, e.g., in the article “The Development of an Instrument to Measure Quality of Vision: The Quality of Vision (QoV) Questionnaire”, by C. McAlinden, K. Pesudovs, and J. E. Moore, published in Investigative Ophthalmology & Visual Science, November 2010, Vol. 51, No. 11, p. 5537, which is incorporated herein in its entirety by reference. This QoV questionnaire asks 30 questions from the patient to evaluate the importance, frequency and severity of ten visual disfunctions on a four-level scale, thus generating the specific patient factors spf(1-i) on a numerical scale, on its way to develop a comprehensive analysis of the quality of vision of the patient.

As an example, a few days after a Light Adjustable Lens (LAL) has been implanted in a patient's eye, the patient may report a deterioration of her visual acuity. The physician may conclude that the LAL shifted away from its intended location during the post-surgical healing process and may decide to adjust the refractive properties of the LAL by illuminating it with a corrective illumination pattern that induces photopolymerization in the LAL. In order to determine what refractive correction is needed, the physician asks the patient to fill out the QoV survey as a patient survey pvt(1) of a patient visual test 155. The patient fills out the QoV survey, and reports 30 responses regarding the various visual disfunctions induced by the shift of the LAL, each on a numerical scale. These 30 numbers are specific patient factors 151 spf(1-j), characterizing the patient's vision. In other embodiments, the patient may give numerical specific patient factors 151 spf(1-1) through spf(1-12) in response to test questions regarding the visual disfunctions as in FIG. 4A. In either embodiment, the specific patient factors 151 spf(i-j) can be inputted into the Prescription Engine 120, which is configured to combine them with diagnostic factors 140 df(k), e.g., the results of an autophoropter measurement, in order to generate a set of refraction modification parameters 130 rmp(j). These refraction modification parameters 130 rmp(j) can represent the refractive correction the patient needs to restore her visual acuity by light-induced photopolymerization of the LAL. For example, the Prescription Engine 120 may combine the received diagnostic factors 140 df(k) from the autophropter measurements with the specific patient factors 151 spf(i-j) from the QoV survey, and in response generate refraction modification parameters 130 rmp(j) that the patient needs a certain amount of optical power addition, and a certain amount of cylinder with a certain cylinder angle. These refraction modification parameters 130 rmp(j) can be inputted into the Refraction Modification System 110, in the present case a Light Delivery Device, which then generates and applies a corrective illumination onto the implanted, shifted LAL to achieve these refractive corrections by appropriate photopolymerization.

FIG. 4B illustrates that when the number of specific patient factors 151 spf(i-j) is too large, it may be unwieldy to enter all of them directly into the Prescription Engine 120 to be combined with the diagnostic factors 140 df(k). To address this issue, some embodiments of the ophthalmic system 100 substantially enhance the utility of patient surveys of visual disfunctions pvt(1), like the QoV questionnaire, by generating much fewer actionable patient factors 150 pf(m) from the large number of specific patient factors 151 spf(i-j). For example, the numerical responses for the 30 questions of a QoV questionnaire, or the specific patient factors 151 spf(1-1)-spf(1-12), can be inputted into a patient factor processor 122 that is configured to combine them into fewer patient factors 150 pf(m). This combination of the specific patient factors 151 pf(i-j) into patient factors 150 pf(m) can be performed by different approaches. The combination can be pre-programmed or can be determined by physician control factors 160 pcf(n). Beyond a simple summation, different importance can be associated with different specific patient factors 151 spf(i-j) by creating a weighted summation of the specific patient factors 151 spf(i-j), by creating a polynomial summation of the specific patient factors 151 spf(i-j), by calculating a higher moment of the specific patient factors 151 spf(i-j), by performing a neural network algorithm, by performing an algorithm using Item Response Theory, or by other known statistics-summarizing methods. In an informative example, in an importance-weighting approach, the physician control factors 160 pcf(n) can be the importance-weighting factors of the approach, and the physician can adjust them based on her/his own diagnosis of the individual patient or based on other patient vision tests 155 pvt(m), described below.

The above descriptions of the ophthalmic system 100 illustrate that the physician control factors 160 pcf(n) can be used at two different stages of the operation of the ophthalmic system 100. First, the physician control factors 160 pcf(n) can be used to impact or control the combining of the individual specific patient factors 151 spf(i-j) into patient factors 150 pf(m) by the patient factor processor 122. Second, the physician control factors 160 pcf(n) can be also used to impact or control the combining of the diagnostic factors 140 df(k) and the patient factors 150 pf(m) to generate the refraction modification parameters 130 rmp(j) for the Refraction Modification System 110. In such embodiments where the physician control factors 160 pcf(n) are only entered into the Prescription Engine 120, the ophthalmic system 100 may not include a patient factor processor 122 and the specific patient factors 151 spf(i-j) can be directly entered into the Prescription Engine 120, thus functioning as the patient factors 150(m) themselves.

In embodiments of the ophthalmic system 100, the patient factor processor 122 can be a self-standing electronic device, for example, an iPad, that runs a code either on board of the patient factor processor 122 or installed in the cloud and run remotely. The patient factor processor 122 can enter the patient factors 150 pf(m) into the Prescription Engine 120 on a direct, wired connection, or via a wireless connection, or through the internet. In other embodiments, the patient factor processor 122 can be part of, or integrated into the Prescription Engine 120, as shown by the dashed line.

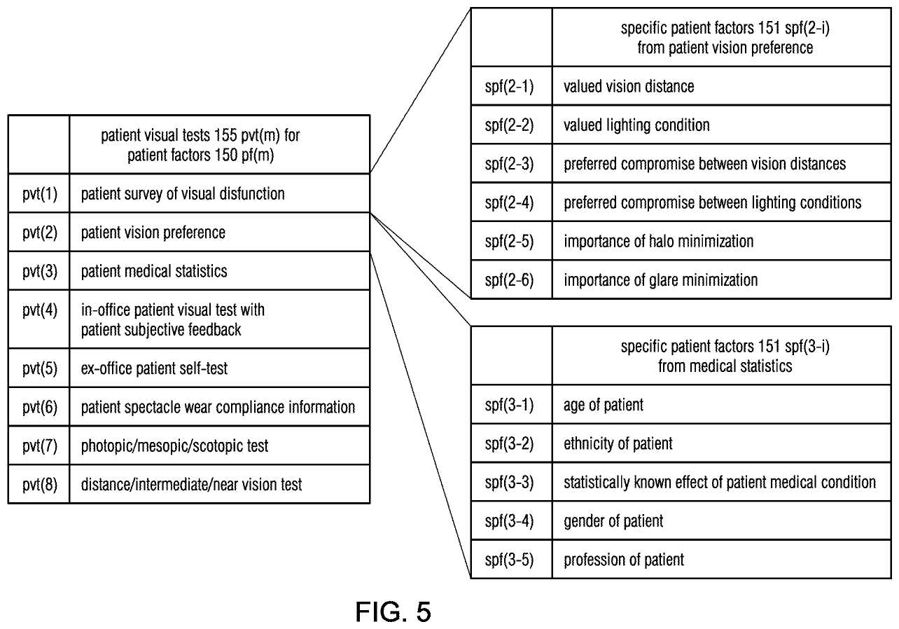

FIG. 5 illustrates embodiments of two other, previously only listed patient vision tests 155: the patient vision preference pvt(2), and the patient medical statistics pvt(3). In some embodiments, it may be reasonable to select the importance-setting weights and techniques used to combine the specific patient factors 151 spf(i-j) into patient factors 150 pf(m) based on patient vision preferences pvt(2). In enlightening examples, a night guard may place a higher than usual value on optimizing his night vision, a truck driver may value her distance vision more highly, and a watch repairman may value optimizing the near vision above all. For such patients, when the specific patient factors 151 spf(1-j) from the patient survey pvt(1) are combined into patient factors 150 pf(m), the physician may increase the weight of the visual outcomes that are highly valued by the patient based on the patient vision preference test pvt(2), and decrease the weight of the specific patient factors 151 spf(1-j) the patient is less concerned about. In another case, a patient whose work requires her to change her vision distance often between near and intermediate vision in a well-lit office, may prefer that a compromise be struck between the results of the near and the intermediate vision test, and the result of this compromise to be treated with a large weight, while the results of the low light (scotopic) vision tests to be treated with a low weight when creating the combined patient factors 150 pf(m).

Other specific patient factors 151 spf(2-i) can be determined with the patient vision preference tests pvt(2), such as the valued vision distance spf(2-1), valued lighting condition spf(2-2), preferred compromise between vision distances spf(2-3), preferred compromise between lighting conditions spf(2-4), importance of halo minimization spf(2-5), importance of glare minimization spf(2-6), or other preferences.

The physician may decide that the patient vision preferences as specific patient factors spf(2-i) are so important that he will enter them directly into the Prescription Engine 120 as patient factors 150 pf(m), to make the most customized decision about the eventual refraction modification. Or, these specific patient factors spf(2-i) can be used to inform the weighing the specific patient factors 151 spf(1-i) gained from a patient survey of visual disfunction pvt(1), as discussed above.

Similarly, patient medical statistical test pvt(3) can also be treated as patient factors 150 pf(m) in some cases. In other cases, patient medical statistical test pvt(3) can impact or control the process of assigning importance to the specific responses to a patient survey pvt(1) in particular. Examples of specific patient factors 151 spf(3-i) that can be determined from a medical statistics test pvt(3) include age of patient spf(3-1), ethnicity of patient spf(3-2), statistically known effects of patient medical condition spf(3-3), gender of patient spf(3-4), and profession of patient spf(3-5), among others. For example, if the patient is a known diabetic, then based on the statistics of medical studies, the physician may foresee that the advancing diabetes will impact the quality of vision differently from a non-diabetic patient, and therefore weigh the specific patient factors 151 spf(1-j) determined by a patient survey pvt(1) differently from the weighing for non-diabetic patients.

In short, the mainly subjective specific patient factors 151 spf(i-j) can be determined from patient visual tests 155 pvt(m) such as patient surveys pvt(1), patient preferences pvt(2), relevant medical statistics pvt(3) and the other test pvt(4)-pvt(8). These specific patient factors 151 spf(i-j) can be used directly as patient factors 150 pf(m) in some embodiments. In others, a patient factor processor 122 can combine these specific patient factors 151 spf(i-j) into patient factors 150 pf(m) in different manners, with different importance factors, impacted by, or under the control of, physician control factors 160 pcf(n).

FIGS. 1A-B, 2, and 6 illustrate that physician-adjustable physician control factors 160 pcf(n) can be used in the ophthalmic system 100 not only to impact the operation of the patient factor processor 122, but also to impact the operation of the Prescription Engine 120 when it combines the dominantly objective diagnostic factors 140 df(k) and the dominantly subjective patient factors 150 pf(m) by the Prescription Algorithm 121.

In embodiments, the one or more physician control factor 160 pcf(n) can be used by the Prescription Algorithm 121 of the Prescription Engine 120 to combine the one or more diagnostic factor 140 df(k) and the one or more patient factor 150 pf(m). Examples of physician control factors 160 pcf(n) include weighting factors in a weighted Prescription Algorithm summation pcf(1), parameters for a non-linear Prescription Algorithm pcf(2); functional settings for a Prescription Algorithm function pcf(3), coding inputs for a Prescription Algorithm code pcf(4), training input for a Machine Learning-based Prescription Algorithm pcf(5), and parameters for a Prescription Algorithm based on Item Response Theory pcf(6). Embodiments of the ophthalmic system 100 provide substantial freedom and control for the physician to choose the relative weight and impact of the dominantly objective diagnostic factors 140 df(k), and the dominantly subjective patient factors 150 pf(m) by judiciously adjusting the physician control factors 160 pcf(n), thereby making the ophthalmic system 100 a particularly powerful and flexible platform to customize and optimize the refraction modification parameters 130 rmp(j) and thereby the refractive adjustments for the individual patient.

FIG. 7 illustrates that in response to the diagnostic factors 140 df(k) and the patient factors 150 pf(m), optionally impacted by the physician control factors 160 pcf(n), the Prescription Engine 120 can output the refraction modification parameters 130 rmp(j), to be used by the Refraction Modification System 110 for its operation. In embodiments, the refraction modification parameters 130 rmp(j) can be an optical power rmp(1), an aberration rmp(2), a coma rmp(3), a cylinder rmp(4), a spherical aberration rmp(5), a Point Spread Function factor rmp(6), and a Modulation Transfer Function factor rmp(7), and several analogous parameters. These refraction modification parameters 130 rmp(j) can represent, for example, the desired refractive outcome, or the desired refractive adjustment to be achieved by the operation of the Refraction Modification System 110.

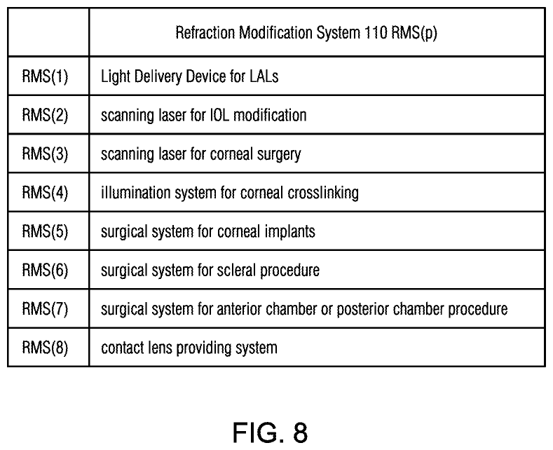

FIG. 8 illustrates that the refraction modification parameters 130 rmp(j) can be coupled into the Refraction Modification System 110 either indirectly, via a physician's action, as in the modular ophthalmic system 100 of FIG. 1A, or directly, as in the integrated ophthalmic system 100 of FIG. 1B. In either case, the Refraction Modification System 110 can be a wide variety of systems, including a Light Delivery Device for Light Adjustable Lenses (LALs) RMS(1), a scanning laser for IOL modification, including the laser-driven formation of refractive or diffractive structures in IOLs RMS(2), a scanning laser for corneal surgery or corneal refraction modification procedure such as LASIK, PRK, and their variants RMS(3), an illumination system for corneal crosslinking RMS(4), a surgical system for corneal implants RMS(5), a surgical system for scleral procedures RMS(6), and a surgical system for anterior chamber or posterior chamber procedures RMS(7). The Refraction Modification System 110 can also be a contact lens providing system RMS(8), or possibly even a spectacle prescription system. It is noted that requirements and implementations of surgical and non-surgical embodiments of the Refraction Modification System 110 can be quite different, given that the effects of the surgical embodiments are hard to reverse, while the effects of the non-surgical embodiments are fully and instantly reversible or adjustable, such as by the removal of the prescription spectacle or contact lens. The visual outcomes of refraction modification procedures by the here-described Refraction Modification System 110 may improve substantially by making use of refraction modification parameters 130 rmp(j) received from the powerful Prescription Engine 120 that combines dominantly objective diagnostic factors 140 df(k) and dominantly subjective patient factors 150 pf(m).

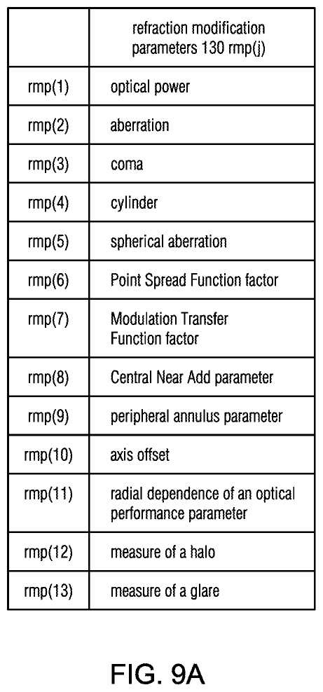

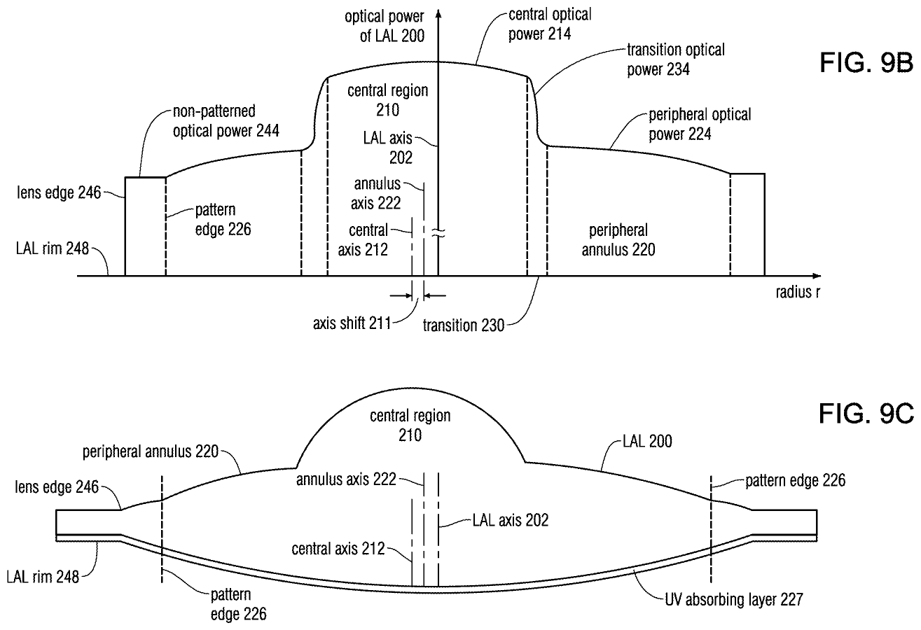

FIG. 9A illustrates the specific embodiment of the ophthalmic system 100, where the Refraction Modification System 110 is a Light Delivery Device (LDD) for illuminating a Light Adjustable Lens (LAL) 200, implanted into the eye. Examples of the Light Delivery Device (LDD) are described in commonly owned U.S. Pat. No. 9,950,482, to Grubbs et al., entitled: “Method for modifying power of light adjustable lens”, hereby incorporated in its entirety by reference. In this embodiment, the refraction modification parameters 130 rmp(j) can again be the previously described optical power rmp(1), aberration rmp(2), coma rmp(3), cylinder rmp(4), spherical aberration rmp(5), Point Spread Function factor rmp(6), or Modulation Transfer Function factor rmp(7).

Beyond the above embodiments, LALs can be also formed with a radially dependent optical power that varies smoothly, giving rise to an Extended Depth Of Focus (EDOF), or has a concentrated Central Near Add (CNA) region with enhanced optical power for near vision, or both. Examples of such EDOF, CNA and EDOF+CNA LALs are described in commonly owned U.S. patent application Ser. No. 16/236,657, to Goldshleger et al., entitled “Blended extended depth of focus light adjustable lens with laterally offset axes”, hereby incorporated in its entirety by reference.

FIG. 9B illustrates the radially varying optical power of such a combined EDOF+CNA LAL 200 as a function of a radius r. The radius can be measured from a LAL axis 202 of the LAL 200. A central, near vision region 210 can have a possibly position-dependent central optical power 214, and a peripheral annulus 220 can have a position-dependent peripheral optical power 224. For optimal visual performance, it is advantageous to center a central axis 212 of the central region 210 on the visual axis of the eye with the iris non-dilated. However, the LAL 200 is typically implanted into the eye and positioned with the iris dilated, thus possibly not being centered with the visual axis of she non-dilated eye. Moreover, the LAL 200 often shifts post-surgically. For both of these reasons, the central axis 212 of the central region 210 may end up shifted relative to the LAL axis 202 and possibly relative to an annulus axis 222 of the peripheral annulus 220 by an axis shift 211. The annulus axis 222 itself may be shifted relative to the LAL axis 202 in embodiments where the peripheral annulus 220 was also formed by an illumination procedure after the implantation of the LAL. Also, the central optical power 214 of the central region 210 can be connected to the peripheral optical power 224 either by a shared, somewhat sharp boundary, as in FIG. 9C, or by a more gradual transition optical power 234 as shown in FIG. 9B. In embodiments, an average of the central optical power 214 can be at least 0.5 diopter different from an average of the peripheral optical power 224. In some embodiments, the average of the central optical power 114 can be at least 1.0 diopter different from the average of the peripheral optical power 124.

FIG. 9C illustrates the position of these regions relative to the physical structure of the LAL 200. Prior to forming the peripheral annulus 220 and the central region 210 in the LAL 200 via illuminations by the LDD, the front and rear surfaces of the LAL 200 typically have a single, approximately constant curvature, and, accordingly, have an optical power that is either independent of the position, or depends on it very weakly, only due to the finite thickness of the LAL 200, for example.

After the LAL 200 is formed by applying a first illumination to form the peripheral annulus 220, and then by applying a second illumination, to form the central region 210, the central axis 212 is often shifted relative to the LAL axis 202, in order to compensate for the postsurgical shift and tilt of the LAL 200, as well as the misalignment of the LAL axis 202 with the visual axis of the eye with the iris in its non-dilated state. Sometime even the annulus axis 222 also ends up being shifted relative to the LAL axis 202.

Within this context, the LALs 200 that have a radially dependent optical power, giving rise to an Extended Depth Of Focus (EDOF), or a Central Near Add (CNA) region, or both, the refraction modification parameters 130 rmp(j) can further include a Central Near Add parameter rmp(8), a peripheral annulus parameter rmp(9), an axis offset/shift rmp(10), a radial dependence of any of the listed optical performance parameters rmp(11), a measure of a halo rmp(12), and a measure of a glare rmp(13). These latter factors are induced in the visual experience by the EDOF or CNA character of this LAL 200.

In some embodiments, the ophthalmic system 100, the Prescription Engine 120 can be configured for generating a warning signal when an inconsistency of a diagnostic factor 140 df(k) and a patient factor 150 pf(m) exceeds a threshold. In a relevant example, in a first step a diagnostic device 145, such as an autorefractor, may forward a set of measured diagnostic factors 140 df(k) to the Prescription Engine 120, directly, or via a physician's action. Next, the physician may be entering into the Prescription Engine 120 the patient factors 150 pf(m) that resulted from an in-office patient visual test pvt(4) that relied on a subjective feedback of the patient, using, for example, a phoropter. The Prescription Engine 120 may discover that one of the objectively determined diagnostic factors 140 df(k), and one of the at least partially subjectively determined patient factors 150 pf(m) may be inconsistent with each other. For example, the patient's visual responses to the phoropter-based vision test may imply an eye refractive power that is significantly different from the refractive power determined by the autorefractor. If the discrepancy, or inconsistency, is large enough, the Prescription Engine 120 may indicate to the physician with a warning signal that at least one of the diagnostic factors 140 df(k) or the patient factors 150 pf(m) need to be determined again.

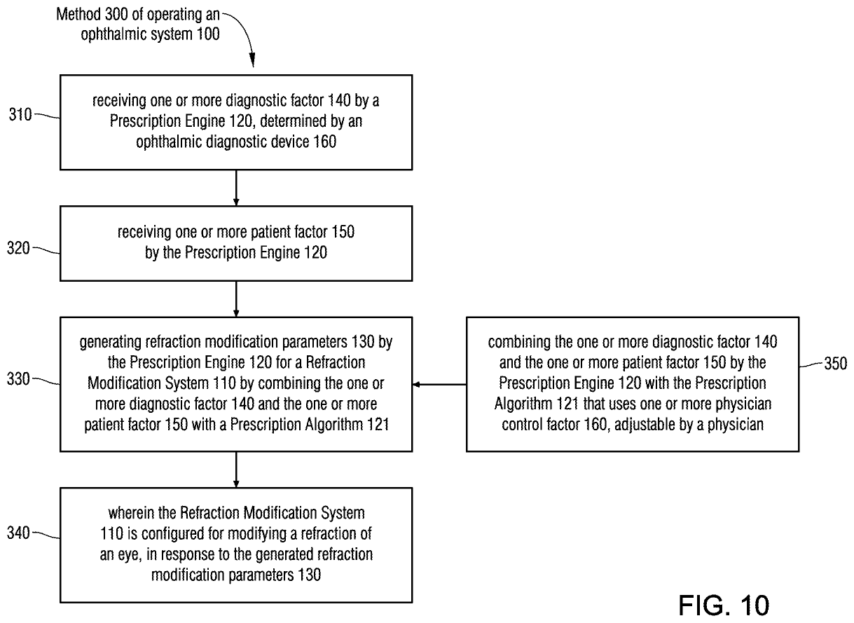

FIG. 10 illustrates a method 300 of operating the ophthalmic system 100, the method 300 comprising:
receiving 310 one or more diagnostic factor 140 df(k) by a Prescription Engine 120, determined by an ophthalmic diagnostic device 145;
receiving 320 one or more patient factor 150 pf(m) by the Prescription Engine 120; and
generating 330 refraction modification parameters 130 rmp(j) by the Prescription Engine 120 for a Refraction Modification System 110 by combining the one or more diagnostic factor 140 df(k) and the one or more patient factor 150 pf(m) with a Prescription Algorithm 121; wherein
the Refraction Modification System 110 is configured for modifying a refraction of an eye, in response to the generated refraction modification parameters 130 rmp(j).

FIG. 3 illustrates that the one or more diagnostic factor 140 df(k) can be a manifest refraction, an optical power df(1), a coma df(2), a cylinder df(3), a spherical aberration df(4), a Point Spread Function factor df(5), or a Modulation Transfer Function factor df(6); and the diagnostic device 145 dd(k) can be a wavefront aberrometer dd(1), an auto refractor dd(2), a Scheimpflug imaging system dd(3), or an Optical Coherence Tomography system dd(4).

FIG. 4A left panel illustrates that the patient factors 150 pf(m) can be determined by a patient vision test 155 pvt(m), which can be a patient survey of visual disfunction pvt(1); a patient vision preference pvt(2); a patient medical statistics pvt(3); an in-office patient visual test involving patient subjective feedback pvt(4); an ex-office patient self-test pvt(5); a patient spectacle wear compliance information pvt(6); a photopic test, a mesopic test, and a scotopic test pvt(7); or a distance vision test, an intermediate vision test, and a near vision test pvt(8). These patient vision test 155 pvt(m) can be performed optionally impacted or controlled by one or more physician control factor 160 pcf(n), adjustable by a physician, as shown in FIG. 4B and detailed earlier and below. In some cases, the ex-office patient self-test pvt(5) can be performed using a portable digital device, a portable electro-optical device, or a mobile phone, among others.

FIG. 4A, right panel shows that the determining the one or more patient factor 150 pf(m) can include determining specific patient factors 151 spf(1-j) by the patient survey related to the visual disfunction pvt(1). As before, the specific patient factors 151 spf(i-j) will be referenced by the corresponding visual disfunction, including low light performance spf(1-1), glare spf(1-2), haloes spf(1-3), blurriness spf(1-4), haziness spf(1-5), starbursts spf(1-6), distortions spf(1-7), double vision spf(1-8), depth perception spf(1-9), fluctuation spf(1-10), focusing difficulty spf(1-11), and a binocular function spf(1-12). Other specific patient factors 151 spf(1-j) can include factors determined by answering questions of a QoV survey.

FIG. 5, right panel shows further specific patient factors 151 spf(2-i), related to patient vision preferences pvt(2); and specific patient factors spf(3-i), related to medical statistics pvt(3), as described earlier.

Returning to FIG. 4B, the determining the one or more patient factor 150 pf(m) can include combining a large number of specific patient factors 151 spf(i-j) into a smaller number of patient factors 150 pf(m) by at least one of a weighted summation; a polynomial summation; a calculation of a higher moment; a neural network algorithm; and an algorithm using Item Response Theory. These combinations can be carried out by the patient factor processor 122 utilizing a pre-programmed code or algorithm. In some cases, the combining by the patient factor processor 122 can include receiving one or more physician control factor 160 pcf(n), adjustable by a physician. In a relevant example, the physician may increase the weight of some of the 30 questions of the QoV survey, described earlier, while decreasing the weight of others.

As shown in FIG. 4B and FIG. 10, a second way the physician control factors 160 pcf(n) can be part of operating the ophthalmic system 100 is that a combining 350 of the one or more diagnostic factor 140 df(k) and the one or more patient factor 150 pf(m) by the Prescription Engine 120 with the Prescription Algorithm 121 uses, or is impacted by one or more physician control factor 160 pcf(n) that are adjusted by a physician.

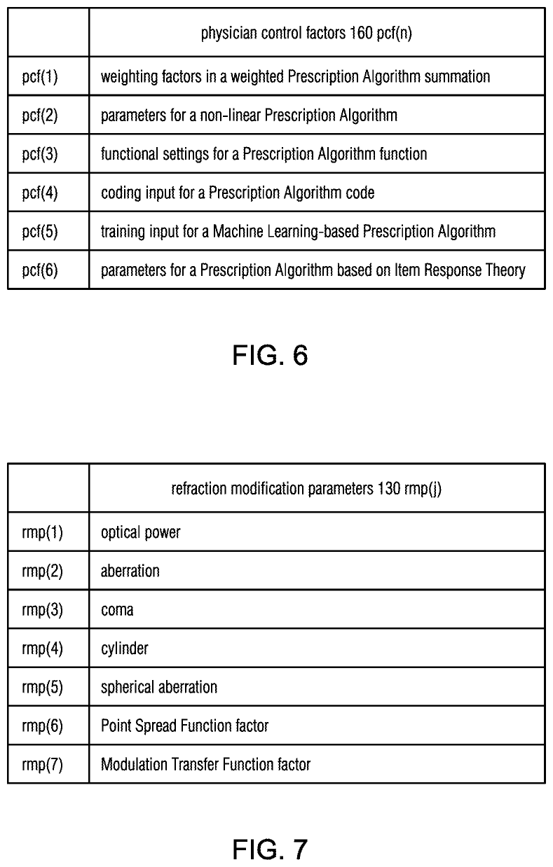

FIG. 6 illustrates that in such embodiments the physician control factors 160 pcf(n), used by the Prescription Algorithm 121 to combine the one or more diagnostic factor 140 df(k) and the one or more patient factor 150 pf(m), can include weighting factors in a weighted Prescription Algorithm summation pcf(1); parameters for a non-linear Prescription Algorithm pcf(2); functional settings for a Prescription Algorithm function pcf(3); coding inputs for a Prescription Algorithm code pcf(4); training input for a Machine Learning-based Prescription Algorithm pcf(5); and parameters for a Prescription Algorithm based on Item Response Theory pcf(6).

The method 300 can include generating a warning signal by the Prescription Engine 120, when an inconsistency of a diagnostic factor 140 df(k) and a patient factor 150 pf(m) exceeds a threshold, as also described earlier.

FIG. 7 illustrates examples of the refraction modification parameters 130 rmp(j) that can be generated by the generating step 330. In response to the diagnostic factors 140 df(k) and the patient factors 150 pf(m), optionally impacted or controlled by the physician control factors 160 pcf(n), the Prescription Engine 120 can output the refraction modification parameters 130 rmp(j) to be used by the Refraction Modification System 110 for its operation. In embodiments, the refraction modification parameters 130 rmp(j) can be an optical power rmp(1), an aberration rmp(2), a coma rmp(3), a cylinder rmp(4), a spherical aberration rmp(5), a Point Spread Function factor rmp(6), and a Modulation Transfer Function factor rmp(7). These refraction modification parameters 130 rmp(j) can represent, for example, the desired refractive outcome, or the desired refractive adjustment to be achieved by the operation of the Refraction Modification System 110.

FIG. 8 illustrates that the method 300 can be practiced in relation to a wide variety of Refraction Modification Systems 110 RMS(p), including a Light Delivery Device for Light Adjustable Lenses (LDD for LALs) RMS(1), a scanning laser for IOL modification, including the laser-driven formation of refractive or diffractive structures in IOLs RMS(2), a scanning laser for corneal surgery or corneal refraction modification procedure such as LASIK, PRK, and their variants RMS(3), an illumination system for corneal crosslinking RMS(4), a surgical system for corneal implants RMS(5), a surgical system for scleral procedures RMS(6), and a surgical system for anterior chamber or posterior chamber procedures RMS(7). The Refraction Modification System 110 can also be a contact lens providing system RMS(8), or possibly even a spectacle prescription system. These latter Refraction Modification Systems 110 can be distinct because of their non-surgical nature.

FIGS. 9A-C illustrate the specific case where the Refraction Modification System 110 is a Light Delivery Device (LDD) for illuminating a Light Adjustable Lens (LAL) 200, implanted into the eye; and the refraction modification parameters 130 rmp(j) can be the previously described optical power rmp(1), aberration rmp(2), coma rmp(3), cylinder rmp(4), spherical aberration rmp(5), Point Spread Function factor rmp(6), or Modulation Transfer Function factor rmp(7).

For LALs 200 that have a radially dependent optical power, giving rise to an Extended Depth Of Focus (EDOF), or a Central Near Add (CNA) region, or both, the refraction modification parameters 130 rmp(j) can further include a Central Near Add parameter rmp(8), a peripheral annulus parameter rmp(9), an axis offset imp(10), a radial dependence of any of the listed optical performance parameters rmp(11), a measure of a halo rmp(12), and a measure of a glare rmp(13). These refraction modification parameters 130 rmp(j) can represent either the desired optical outcome, or the desired optical adjustment of the LAL. Details of the embodiments of EDOF+CNA LALs 200 have been described earlier.

While this document contains many specifics, details and numerical ranges, these should not be construed as limitations of the scope of the invention and of the claims, but, rather, as descriptions of features specific to particular embodiments of the invention. Certain features that are described in this document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed, combination may be directed to another subcombination or a variation of a subcombination.

1. An ophthalmic system, comprising:
a Refraction Modification System for modifying a refraction of an eye, in response to refraction modification parameters; and
a Prescription Engine, for generating the refraction modification parameters for the Refraction Modification System by a Prescription Algorithm that combines
one or more diagnostic factor, determined by an ophthalmic diagnostic device, and
one or more patient factor.

2. The ophthalmic system of claim 1, wherein:
the ophthalmic system is modular such that the Prescription Engine, separate from the Refraction Modification System, is configured for outputting the refraction modification parameters; and
the Refraction Modification System is configured for receiving the refraction modification parameters through a physician action.

3. The ophthalmic system of claim 1, wherein:
the ophthalmic system is integrated such that the Prescription Engine is configured for coupling the refraction modification parameters into the Refraction Modification System, optionally under a control of a physician.

4. The ophthalmic system of claim 1, wherein:
the one or more diagnostic factor is selected from the group consisting of a manifest refraction, an optical power, a coma, a cylinder, a spherical aberration, a Point Spread Function factor, and a Modulation Transfer Function factor.

5. The ophthalmic system of claim 1, wherein:
the diagnostic device is selected from the group consisting of a phoropter, a wavefront aberrometer, an auto refractor, a Scheimpflug imaging system, and an Optical Coherence Tomography system.

6. The ophthalmic system of claim 1, wherein:
one or more patient factor is determined by a patient vision test, selected from
a patient survey of visual disfunctions;
a patient vision preference;
a patient medical statistics;
an in-office patient visual test involving patient subjective feedback;
an ex-office patient self-test;
a patient spectacle wear compliance information;
a photopic test, a mesopic test, and a scotopic test; and
a distance vision test, an intermediate vision test, and a near vision test;
optionally impacted by one or more physician control factor, adjustable by a physician.

7. The ophthalmic system of claim 6, wherein:
specific patient factors are determined by the patient survey related to visual disfunction selected from a group consisting of
low light performance, glare, haloes, blurriness, haziness, starbursts, distortions, double vision, depth perception, fluctuation, focusing difficulty, and a binocular function.

8. The ophthalmic system of claim 7, wherein:
specific patient factors are combined into patient factors by at least one of
a weighted summation;
a polynomial summation;
a calculation of a higher moment;
a neural network algorithm; and
an algorithm using Item Response Theory,
optionally impacted by one or more physician control factor, adjustable by a physician.

9. The ophthalmic system of claim 1, wherein:
the Prescription Engine is configured for combining the one or more diagnostic factor and the one or more patient factor by the Prescription Algorithm that uses one or more physician control factor, adjustable by a physician.

10. The ophthalmic system of claim 9, wherein:
the one or more physician control factor is used by the Prescription Algorithm to combine the one or more diagnostic factor and the one or more patient factor, as at least one of
weighting factors in a weighted Prescription Algorithm summation;
parameters for a non-linear Prescription Algorithm;
functional settings for a Prescription Algorithm function;
coding inputs for a Prescription Algorithm code;
training input for a Machine Learning-based Prescription Algorithm; and
parameters for a Prescription Algorithm based on Item Response Theory.

11. The ophthalmic system of claim 1, wherein:
the refraction modification parameters are selected from the group consisting of
an optical power, an aberration, a coma, a cylinder, a spherical aberration, a Point Spread Function factor, and a Modulation Transfer Function factor.

12. The ophthalmic system of claim 1, wherein:
the Refraction Modification System is selected from the group consisting of
a scanning laser for IOL modification, a scanning laser for corneal surgery, an illumination system for corneal crosslinking, a surgical system for corneal implants, a surgical system for scleral procedures, a surgical system for anterior chamber or posterior chamber procedures, and a contact lens providing system.

13. The ophthalmic system of claim 1, wherein:
the Refraction Modification System is a Light Delivery Device, for illuminating a Light Adjustable Lens implanted into the eye; and
the refraction modification parameters are selected from the group consisting of
an optical power, an aberration, a coma, a cylinder, a spherical aberration, a Point Spread Function factor, and a Modulation Transfer Function factor, a Central Near Add parameter, a peripheral annulus parameter, an axis offset, a radial dependence of any of the optical performance parameters listed here, a measure of a halo, and a measure of a glare.

14. The ophthalmic system of claim 1, wherein:
the Prescription Engine is configured for generating a warning signal, when an inconsistency of a diagnostic factor and a patient factor exceeds a threshold.

15. A method of operating an ophthalmic system, the method comprising:
receiving one or more diagnostic factor by a Prescription Engine, determined by an ophthalmic diagnostic device;
receiving one or more patient factor by the Prescription Engine; and
generating refraction modification parameters by the Prescription Engine for a Refraction Modification System by combining the one or more diagnostic factor and the one or more patient factor with a Prescription Algorithm; wherein
the Refraction Modification System is configured for modifying a refraction of an eye, in response to the generated refraction modification parameters.

16. The method of claim 15, wherein:
the one or more diagnostic factor is selected from the group consisting of a manifest refraction, an optical power, a coma, a cylinder, a spherical aberration, a Point Spread Function factor, and a Modulation Transfer Function factor; and
the diagnostic device is selected from the group consisting of a wavefront aberrometer, an auto refractor, a Scheimpflug imaging system, and an Optical Coherence Tomography system.

17. The method of claim 15, comprising:
determining one or more a patient factor by a patient vision test from at least one of
a patient survey of visual disfunction;
a patient vision preference;
a patient medical statistics;
an in-office patient visual test involving patient subjective feedback;
an ex-office patient self-test;
a patient spectacle wear compliance information;
a photopic test, a mesopic test, and a scotopic test; and
a distance vision test, an intermediate vision test, and a near vision test;
optionally impacted by one or more physician control factor, adjustable by a physician.

18. The method of claim 17, the determining the one or more patient factor comprising:
determining specific patient factors by the patient survey related to the visual disfunction, selected form a group consisting of
low light performance, glare, haloes, blurriness, haziness, starbursts, distortions, double vision, depth perception, fluctuation, focusing difficulty, and a binocular function.

19. The method of claim 18, the determining the one or more patient factor comprising:
combining specific patient factors into patient factors by at least one of
a weighted summation;
a polynomial summation;
a calculation of a higher moment;
a neural network algorithm; and
an algorithm using Item Response Theory,
optionally impacted by one or more physician control factor, adjustable by a physician.

20. The method of claim 17, wherein:
the ex-office patient self-test is performed using at least one of a portable digital device, a portable electro-optical device, and a mobile phone.

21. The method of claim 15, wherein the combining comprises:
combining the one or more diagnostic factor and the one or more patient factor by the Prescription Engine with the Prescription Algorithm that uses one or more physician control factor, optionally adjustable by a physician.

22. The method of claim 21, wherein:
the one or more physician control factor is used by the Prescription Algorithm to combine the one or more diagnostic factor and the one or more patient factor, as at least one of
weighting factors in a weighted Prescription Algorithm summation;
parameters for a non-linear Prescription Algorithm;
functional settings for a Prescription Algorithm function;
coding inputs for a Prescription Algorithm code;
training input for a Machine Learning-based Prescription Algorithm; and
parameters for a Prescription Algorithm based on Item Response Theory.

23. The method of claim 15, wherein:
the Refraction Modification System is a Light Delivery Device, for illuminating a Light Adjustable Lens implanted into the eye; and
the refraction modification parameters are selected from the group consisting of
an optical power, an aberration, a coma, a cylinder, a spherical aberration, a Point Spread Function factor, and a Modulation Transfer Function factor, a Central Near Add parameter, a peripheral annulus parameter, an axis offset, a radial dependence of any of the optical performance parameters listed here, a measure of a halo, and a measure of a glare.

24. The method of claim 15, comprising:
generating a warning signal by the Prescription Engine, when an inconsistency of a diagnostic factor and a patient factor exceeds a threshold.
